(19) United States
(12) Patent Application Publication
(10) Pub. No.: US 2016/0030207 A1
(43) Pub. Date: Feb. 4, 2016

(54) PROSTHESIS COOLING SYSTEM

(71) Applicant: LETO SOLUTION, INC., San Antonio, TX (US)

(72) Inventors: Gary Lynn Walters, JR., San Antonio, TX (US); Jake Parker Montez, Corpus Christi, TX (US); David Konrad Schultz, Kerrville, TX (US); Austin Jeffrey Darius, San Antonio, TX (US)

(21) Appl. No.: 14/377,899

(22) PCT Filed: Apr. 28, 2014
(86) PCT No.: PCT/US14/35659

§ 371 Date: Aug. 11, 2014

Related U.S. Application Data
(60) Provisional application No. 61/816,570, filed on Apr. 26, 2013.

(51) Int. Cl. A61F 2/80

(52) U.S. Cl. CPC A61F 2/80

(57) ABSTRACT

A prosthesis cooling system may comprise one or more thermoelectric cooling devices embedded in a prosthesis socket, a power source, and a power control circuit. The cooling system may use active or passive cooling, and may use a closed-loop temperature control system.

### CROSS-REFERENCE TO RELATED APPLICATIONS

This application claims priority to U.S. Provisional Patent Application Ser. No. 61/816,570 filed Apr. 26, 2013, the entire disclosure of which is herein incorporated by reference.

### BACKGROUND

Amputees may use a variety of prostheses designed to replace a residual limb. The residual limb may include bone, muscle, tissue and skin. For example, a leg amputee may use a prosthetic leg, and an arm amputee may use a prosthetic arm. Prosthetic limbs generally include a socket and liner designed to fit over a residual limb. The socket may be a relatively rigid shell that encases a residual limb. The liner may provide a flexible cushion between the residual limb and socket, and act as a “second skin” between the residual limb and socket. Ideally, the liner will provide a consistent link between the residual limb and socket such that the prosthesis can move as one with the residual limb through a variety of motions.

Liners are generally selected by a prosthetist to provide a high-quality interface between the residual limb and socket. In selecting a suitable liner, a prosthetist may consider, for example, the shape and condition of the residual limb, patient activity level, socket design, patient functional needs and type of suspension system, among other factors. Liners may comprise one or more materials, such as silicone, urethane, copolymer, gel, thermoplastic elastomers, textiles and other suitable materials. For example, a liner may comprise an inner urethane layer and an outer textile layer. Liners may further comprise a variety of thicknesses.

Liners may also include non-stick treatments, and may include internal matrices to promote stability. Liners may include a variety of additives, such as emollients, anti-bacterial compounds, antioxidants and other skin-friendly substances. Liners may be replaced more frequently than sockets, and the frequency of replacement may depend on the material used and patient condition. Typically, a liner may be replaced every six months.

Similarly, sockets may comprise a variety of materials, such as resin, acrylic, carbon fiber, and other suitable materials. Sockets may comprise one or more rigid layers, such as acrylic and/or carbon fiber. Also, in some cases, a flexible inner socket may be used between the hard socket and liner so as to provide a better connection with more dynamic linkage between the residual limb and the prosthesis. An inner socket may provide a greater surface area for connection with a rigid socket, an elevated vacuum and reduced friction in the socket. Various prosthetic components may be affixed to the socket, such as pylons, rotators, joints, a foot, a hand or other terminal device. In addition to replacement for wear and damage, sockets may be replaced as needed to accommodate physical growth, changes in the residual limb, or new suspensions or terminal devices, new socket materials, and patient condition. Typically, a socket may be replaced every two to five years

Despite advances in prosthesis fit and comfort, liners and sockets are built from materials that are generally not gas permeable, nor do they allow significant transfer heat away from the residual limb, or at a rate sufficient to prevent sweating during times of increased environmental temperature or activity. The prosthesis may prevent or hinder the body's natural dissipation of heat through the residual limb. When the residual limb temperature increases to or beyond the sweat threshold, sweat may accumulate between the skin and liner. Sweat accumulation leads to limb degradation and poor linkage between the residual limb and prosthesis. There remains a need for a suitable system and method for removing excess heat from a residual limb wholly or partially encased in a liner and socket.

### SUMMARY

A prosthesis cooling system comprising one or more thermoelectric cooling elements (TECs) configured for connecting to the socket of a prosthetic limb; and a power source electrically coupled to each TEC.

A cooling system for a transtibial (BK) prosthesis comprising a socket, the system comprising one or more TECs configured for connecting to the socket at a location generally corresponding to a first apex of the gastrocnemius muscle; and a power source electrically coupled to each TEC.

A prosthesis comprising a socket and cooling system, the cooling system comprising at least one TEC connected to the socket of a prosthetic limb; and a power source electrically coupled to the at least one TEC.

A method of mounting a TEC to the socket of a prosthesis, the method comprising forming an aperture in the socket, the aperture being configured to receive a TEC comprising a cold side surface, a hot side surface and leads; and mounting the TEC to the aperture such that the cold side surface faces the interior of the socket and the hot side surface faces the atmosphere.

A cooling system for an above-knee (AK) prosthesis comprising a socket, the system comprising a first TEC configured for embedding in the socket at a location generally corresponding to a location between the rectus femoris and vastus lateralis; a second TEC configured for embedding in the socket at a location generally corresponding to a location between the rectus femoris and the vastus medialis; a third TEC configured for embedding in the socket at a location generally corresponding to a location near the biceps femoris and the femoral artery; and a power source electrically coupled to each TEC.

### BRIEF DESCRIPTION OF THE DRAWINGS

FIG. 1 illustrates an embodiment of a prosthesis having a prosthesis cooling system mounted thereto.

FIG. 2 illustrates an embodiment of a socket wall configured for mounting a TEC.

FIG. 3 illustrates an embodiment of a TEC embedded in a prosthesis socket.

FIG. 4 illustrates another embodiment of a TEC embedded in a prosthesis socket.

FIG. 5 illustrates an embodiment of energy charts for an exemplary TEC.

FIG. 6 provides an example of TEC placement for a BK right-leg prosthesis.

FIG. 7 provides an example of TEC placement for a BK right-leg prosthesis.

FIG. 8 illustrates an embodiment of a prosthesis socket having an enclosure for containing a PCM packet.

FIG. 9 illustrates an embodiment of an enclosure for a PCM packet.

FIG. 10 illustrates an example state transition diagram of a prosthesis cooling system.

FIG. 11 illustrates an example functional block diagram of a control system.

FIG. 12 illustrates an example of TEC placement for an AK right-leg prosthesis.

### DETAILED DESCRIPTION

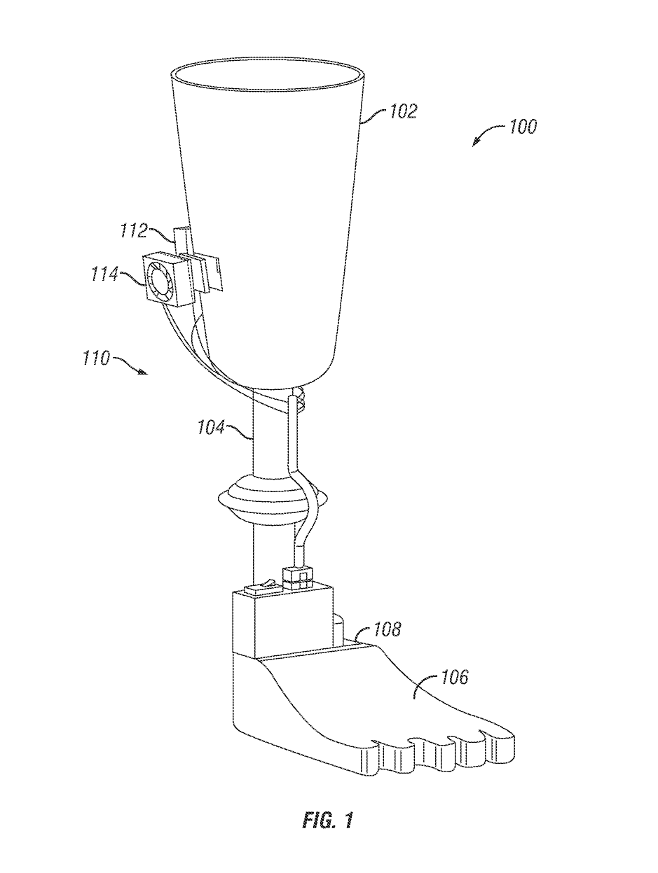

The embodiment of FIG. 1 is disclosed in connection with an example prosthesis for a transtibial (below-knee or BK) amputee. As may be seen in the embodiment of FIG. 1, the prosthesis 100 may include a socket 102, pylon 104 and foot 106. A vacuum suspension system 108 may be used to assist the wearer in retaining prosthesis linkage to the residual limb (not shown).

A prosthesis cooling system 110 may be mounted to the prosthesis 100. In the embodiment of FIG. 1, the prosthesis cooling system 110 may include a thermoelectric cooling element (TEC, or Peltier device) 112 embedded in or otherwise mounted to the socket 102 of a prosthesis 100. The TEC 112 may comprise a cold side surface and a hot side surface on opposing sides of the TEC 112. The TEC may be embedded or mounted to the socket 102 such that the cold side surface may receive heat from the residual limb. The hot side surface may be oriented to directly or indirectly dissipate heat to atmosphere. In such embodiments, the cold side surface may generally face the interior of the socket where a residual limb may be disposed, and the hot side surface may be exposed to atmosphere or thermally coupled to a heat sink and/or active cooling device 114, such as a fan. The TEC 112 may be powered by any suitable power source, such as a battery or fuel cell (not shown).

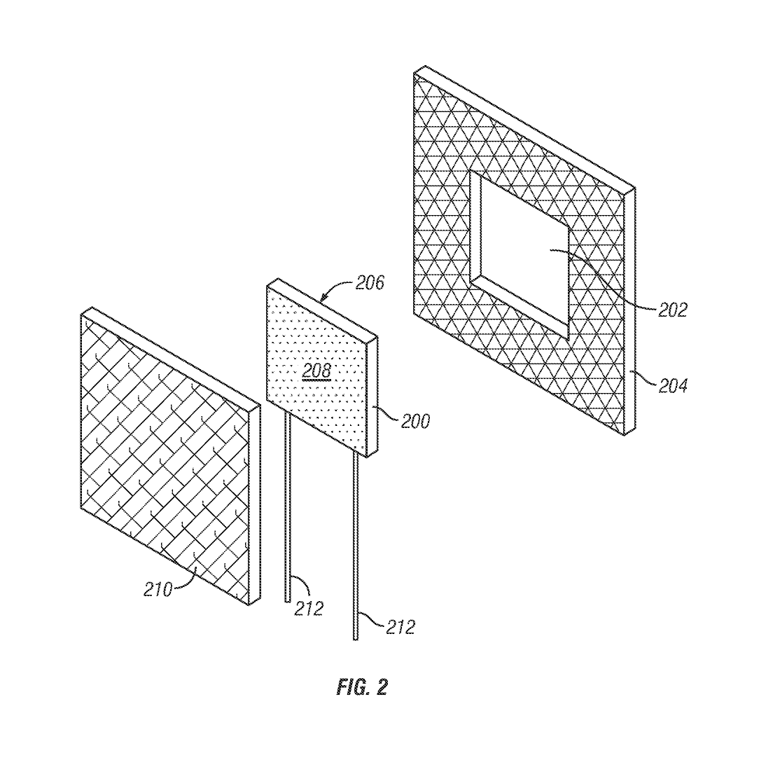

A TEC may, in some embodiments, be embedded in the socket wall. As may be seen in the embodiment of FIG. 2, a TEC 200 may be mounted in an aperture 202 of the socket wall 204. The cold side surface 206 of the TEC may face the residual limb. The hot side surface 208 may face toward or remain outside of the socket. A heat sink 210 may be mounted to or made as part of the hot side surface 208 so as to increase the surface area for heat dissipation. In some embodiments, the heat sink 210 may be mounted to the hot side surface 208 using thermal epoxy, silicone RTV, weldment, or other any suitable means. The TEC leads 212 may be connected to a power source.

Prosthetic limbs are generally custom-made for a residual limb by a prosthetist. A prosthetist may evaluate the size and condition of the residual limb, and patient health and lifestyle, among other things, in designing a suitable prosthesis. Such design may include estimating the amount of heat loss through the residual limb for various patient activities, as discussed in more detail below. Based on the estimated heat loss across a range of activities and the particular prosthesis and residual limb, a prosthetist may select a cooling system as described herein for inclusion in the prosthesis during the manufacturing process. In other embodiments, a cooling system may be fitted to an already-built prosthesis.

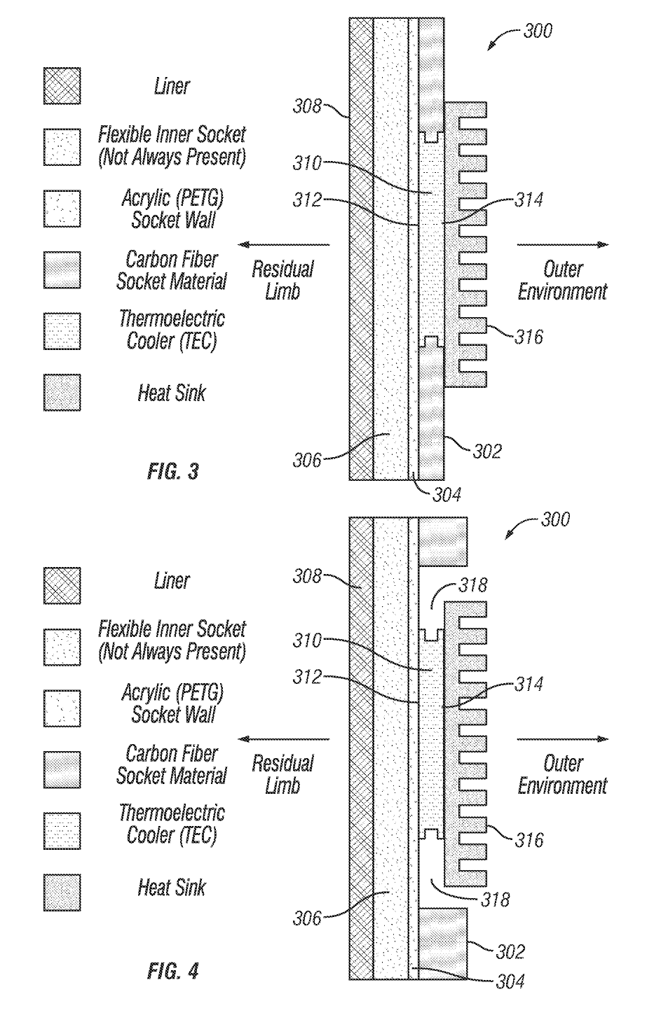

FIGS. 3 and 4 illustrate other embodiments of TEC embedding or mounting. In the embodiment of FIG. 3, for example, a socket 300 may comprise a relatively rigid carbon fiber outer layer 302, an acrylic middle layer 304, and a more flexible inner socket 306. A liner 308 may be disposed in the socket 300 inside inner socket 306. A flexible inner socket may be optionally used with a rigid socket. A TEC 310 may be embedded in the carbon fiber layer 302 of the socket 300. The cold side surface 312 of the TEC may generally face the residual limb. The hot side surface 314 may face the atmosphere. In the embodiment of FIG. 3, the TEC cold side surface 312 may interface with the acrylic layer 304. A heat sink 316 may be mounted to the hot side surface 314 of the TEC. As may be seen in the embodiment of FIG. 3, the carbon fiber layer 316 may abut all or part of the TEC 310, and the heat sink 316 may overlap the carbon fiber layer 302 to create a more pleasing appearance and seal the TEC from environmental contamination. And as may be seen in the alternate embodiment of FIG. 4, some space 318 may remain between the TEC 310 and the carbon fiber layer 302 so as to accommodate a thicker carbon fiber layer 302 and promote further heat dissipation.

Of course, the TEC may be mounted to the socket in a variety of ways, depending on socket configuration and patient needs. Some sockets may have different layers, may have only a single layer, or may have voids or structural elements incorporated into the socket design. For example, a TEC may be mounted such that the cold side surface is exposed to the interior of the socket, and may interface directly with a liner or inner socket. In other embodiments, the TEC cold side surface may be separated from the interior of the socket by one or more layers.

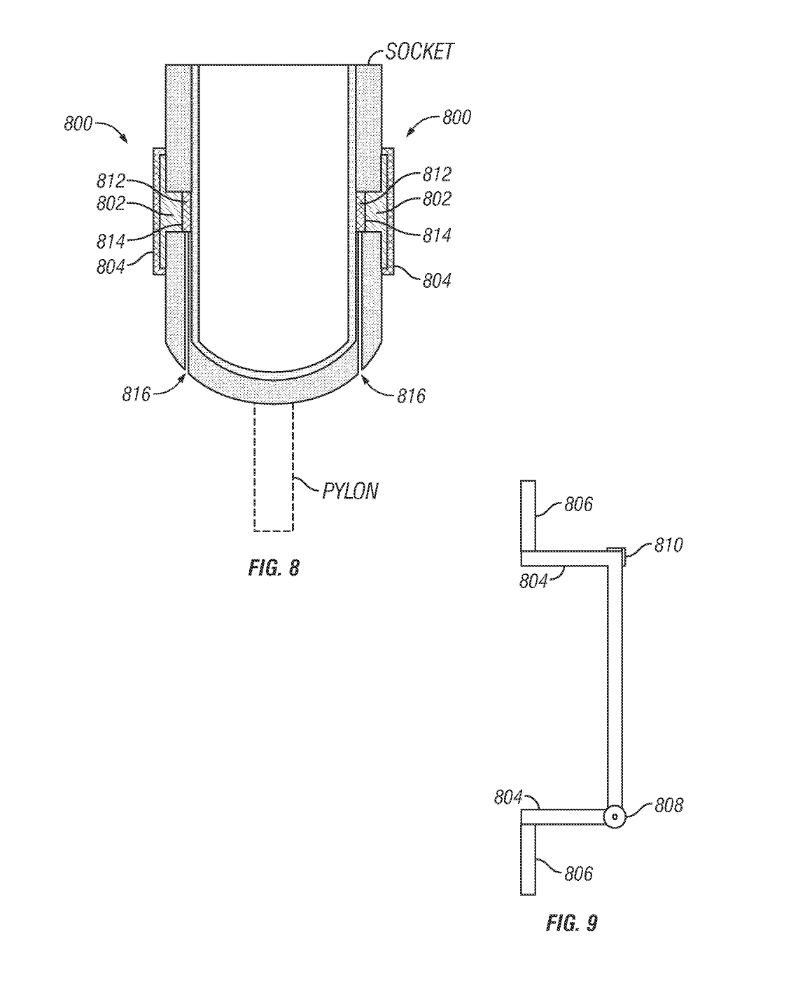

During socket fabrication, a prosthetist may use a dummy TEC or plastic model or frame to form apertures in the desired socket layer(s) to receive a TEC. A TEC may be mounted in the aperture and bonded in place with a suitable bonding agent, such as thermal epoxy. Likewise, small-diameter tubing may be embedded in one or more of the socket layers to provide channels 816 (with reference to FIG. 8) for routing TEC wire leads.

Similarly, more than one TEC may be used for any given prosthesis and placed so as to optimize heat dissipation and the health of the residual limb. In a BK prosthesis, for example, a TEC may be mounted generally over major calf muscles that may remain as part of the residual limb. In some embodiments, TECs may be embedded in the socket in the regions where the lateral and posterior apexes of the gastrocnemius muscle group of the residual limb would be disposed within the socket.

An analysis of heat generation for a residual limb (e.g., a BK residual limb), may reveal how much heat must be removed from the region covered by a prosthesis, such as may be shown from a variety of studies, e.g., Klute, Glenn K., “Residual-Limb skin temperature in transtibial Sockets” Journal of Rehabilitation Research & Development March/April 2005 Volume 42, Number 2, Pages 147-154; Cross, A., Collard, M., Nelson, A., “Body Segment differences in Surface Area, Skin Temperature and 3D Displacement and the Estimation of Heat Balance during Locomotion in Hominins” PLoS ONE; Klute, Glenn K., “A Three-Dimensional Finite Element Model of the Transtibial Residual Limb and Prosthetic Socket to Predict Skin Temperatures” IEEE Transactions on Neural systems and Rehabilitation Engineering September 2006 Volume 14, Number 3, Pages 336-342; and Klute, Glenn K., “The Thermal Conductivity of Prosthetic Sockets and Liners” Prosthetics and Orthotics international September 2007 Volume 31, Number 3, Pages 292-299. For example, a lower limb (below the knee) may include approximately 7% of the total body surface area, and may have a heat loss of approximately 8.5 watts from natural cooling modes (convection, radiation, and conduction—mostly convection and radiation). A prosthesis cooling system may be designed to compensate for loss of natural cooling when a residual limb is encased in a prosthesis. A BK amputee retaining certain gastrocnemius muscle mass may thus be calculated to require up to 9 watts of body waste heat removal.

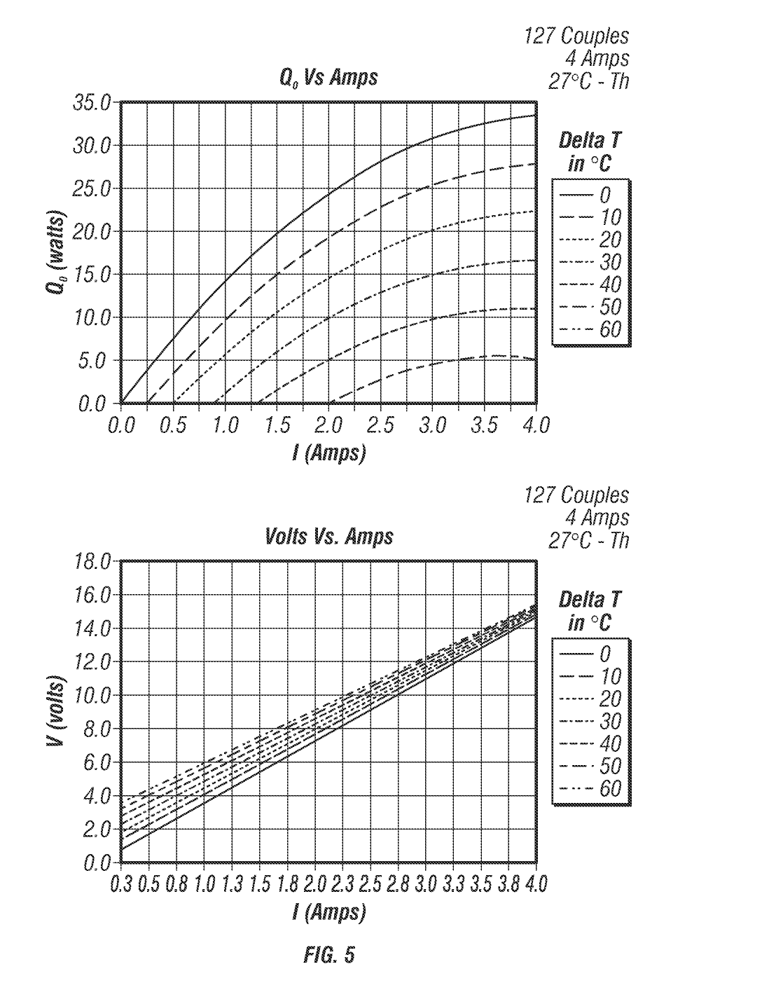

A suitable system comprising one or more TECs may be thus designed based on TEC energy characteristics, such as those of the exemplary power curves of FIG. 5. Inefficiencies of the TEC may be also considered when selecting a TEC and heat sink. For example, if a TEC itself generates 9 watts of waste heat through inefficiency while removing 9 watts of body wast heat, a heat sink having a capacity of at least 18 watts may be desired. In one embodiment, given prosthesis size and power source constraints, a 25.7-watt, 3.0 Aₘₐₓ, 15.4Vₘₐₓ TEC may be selected.

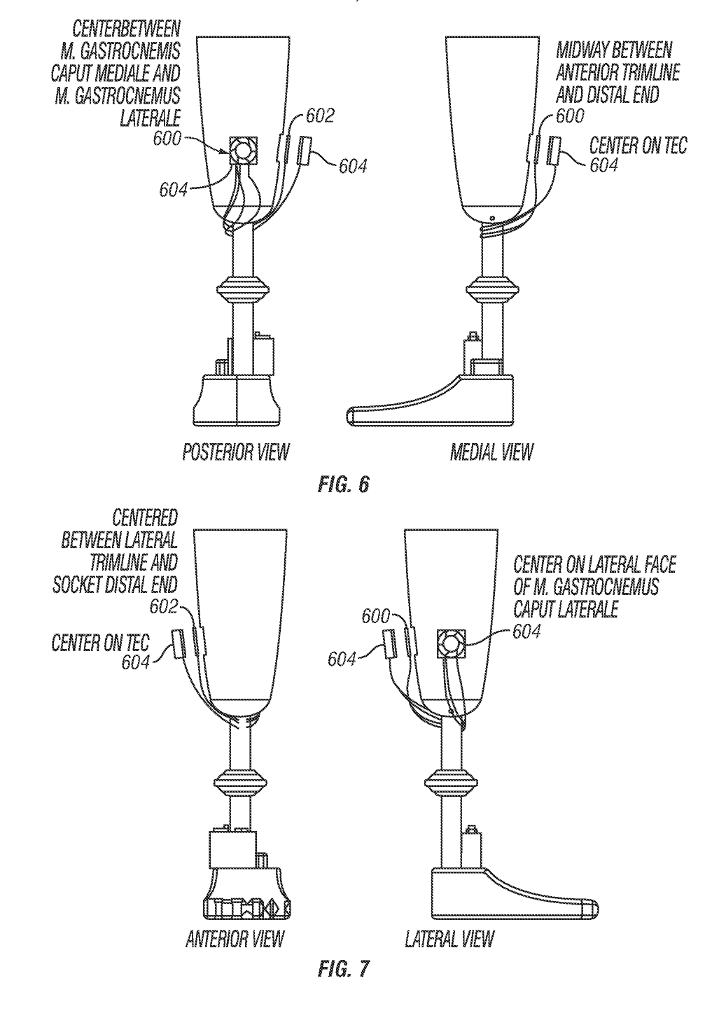

Further, more than one TEC may be used to increase the rate of body waste heat removal. Using more than one TEC may also reduce the amount of body waste heat that each TEC must remove, which may allow each to run at a lower power level and generate less waste heat through inefficiency. Thus, a smaller heat sink may be used for each TEC. As may be seen in the embodiment of FIGS. 6 and 7, a TEC 600 may be generally centered over the medial calf face, and a TEC 602 may be generally centered over the lateral calf face for a right-leg BK prosthesis. As may be suitable for the residual limb, a prosthetist may position each TEC 600 and 602 to allow for body waste heat removal by the TECs. One or more actively cooled (fan-cooled) heat-sinks 604 may be used to assist in waste heat removal.

Each TEC may or may not be placed for optimal cooling. Generally, optimal cooling may suggest placement of TEC in a prosthesis socket over the centerline of the muscle mass. However, TEC cooling efficiency may be balanced against accommodation of patient activity, among other things. For example, placing a TEC at the anterior and posterior centerlines of a socket may result in interference with certain basic patient activities, such as sitting, and moving from a standing position to a seated position, and back to a standing position. TEC placement at such positions may cause the TECs to interfere to an undesirable degree with furniture, patient comfort and clothing, particularly if the TEC is used in connection with an active heat sink. Accordingly, one or more TECs may be placed so as to accommodate expected patient activity. Thus, in some embodiment, such as described in more detail below in connection with AK prostheses, TECs may be offset from muscle mass centerlines.

In one embodiment, ten minutes of exercise may raise the temperature in the BK residual limb to approximately 98° F. at the posterior region, which temperature is typically above the sweat threshold. With the foregoing TEC configuration running at 6V, temperature may be reduced by approximately 2° F., resulting in noticeable increase in patient comfort. If running at 7.6V, the cooling system may reduce the temperature by 5° F. to below the typical sweat threshold. Such cooling may thus reduce or eliminate sweat production and accumulation, thus promoting the health of the residual limb.

For example, analyzing the cooling load for a prosthesis cooling system may involve calculating heat diffusion in a conductor such as that of Equation 1:

$$ρ {C}_{p} \frac{\partial T}{\partial t} = Q + ∇ \cdot ( k ∇ T ) \qquad Equation 1$$

A heat analysis model may consider all elements of the heat diffusion equation for a cylinder. A fully developed three-dimensional and transient heat model may adequately reflect heat generation in a residual limb, e.g., on a real-time basis. Cylindrical coordinates may be selected for ease of calculation, as may be seen in Equation 2:

$$ρ {C}_{p} \frac{\partial T}{\partial t} = \frac{1}{r} \frac{\partial }{\partial r} ( kr \frac{\partial T}{\partial r} ) + \frac{1}{{r}^{2}} \frac{\partial }{\partial ϕ} ( k \frac{\partial T}{\partial ϕ} ) + \frac{\partial }{\partial z} ( k \frac{\partial T}{\partial x} ) + \dot{q} \qquad Equation 2$$

Material properties of the prosthesis may be considered. For example, a urethane liner may be isotropic, thus permitting elimination of non-uniform temperature distribution. Such an assumption may reduce the heat diffusion equation of Equation 2 to radial and angular position terms, as may be seen in Equation 3:

$$ρ {C}_{p} \frac{\partial T}{\partial t} = \frac{1}{r} \frac{\partial }{\partial r} ( kr \frac{\partial T}{\partial r} ) + \frac{1}{{r}^{2}} \frac{\partial }{\partial ϕ} ( k \frac{\partial T}{\partial ϕ} ) + \dot{q} \qquad Equation 3$$

A residual limb, after exercise, may be modeled as continuing into an indefinite transient state of heat generation. Under such an assumption, the transient term in the foregoing heat diffusion Equation 3 may be considered steady state. Such an approach may serve to simplify numerical analysis in the residual limb, allowing for more rapid determination of a suitable configuration of a prosthesis cooling system based on a model residual limb having a generalized shape and condition. Thus, as may be seen in Equation 4, temperature on a basis of radius and angular position may be related to the internal heat generation of the model residual limb:

$$- \dot{q} = \frac{1}{r} \frac{\partial }{\partial r} ( kr \frac{\partial T}{\partial r} ) + \frac{1}{{r}^{2}} \frac{\partial }{\partial ϕ} ( k \frac{\partial T}{\partial ϕ} ) \qquad Equation 4$$

To develop additional parameters for time to cooling and heat absorption required for a TEC, one may use Newton's Law of Cooling, as may be seen in Equation 5:

T(t)=Tₐ+(Tₒ−Tₐ)e^{-kt} Equation 5

Likewise, the First Law of Thermodynamics may be used, as may be seen in Equation 6:

Q=mCₚtΔT Equation 6

In order to dissipate the heat generated by perfusion and metabolic processes in the residual limb, one or more TECs may be mounted to the prosthesis so as to facilitate an acceptable level of heat flux \dot{Q}_{c} away from the socket interior environment. These TECs may require a heat sink capable of rejecting heat from both the heat generated by the residual limb and the resistive heating by the thermoelectric element, \dot{Q}_{gen}. \dot{Q}_{gen} may, in some cases, be obtained from a manufacturing datasheet provided by the TEC manufacturer (FIG. 5). the total heat flux \dot{Q}ₜ may be calculated using Equation 7:

\dot{Q}ₜ=\dot{Q}_{c}+\dot{Q}_{gen} Equation 7

As in an embodiment described herein, an estimated \dot{Q}ₜ≅15 W per TEC may be calculated. Because a hot surface of a heat sink could potentially be in contact with human skin and thus present a burn risk, an upper limit of T_{f}=150° F. may be used as a constraint to prevent burns and enhance the overall safety of the prosthesis cooling system. General environmental conditions may be considered as well. For example, a hot summer day may be considered. In such an embodiment, for example, an atmospheric temperature T_{∞} may be constrained conservatively at 110° F. Heat flux Equation 8 may be rearranged to calculate thermal resistance (R), which in this embodiment yields a required thermal resistance R of approximately 2.7° C./W or lower.

$$\dot{Q} = \frac{Δ T}{R} \qquad Equation 8$$

If an active heat sink is not desired, then a heat transfer analysis may assume natural convection conditions in combination with radiation, as in Equation 9:

\dot{Q}ₜ=\dot{Q}_{conv}+\dot{Q}_{rad} Equation 9

A variety of heat sink configurations may be evaluated. Heat sinks may vary substantially by material, fin count and configuration, dimension, and other properties. Heat transfer capabilities of various heat sinks may therefore depend on geometry and any constrained temperature value (e.g., atmospheric temperature). As a corollary to the assumption of natural convection, a relatively low heat transfer coefficient h may be obtained (e.g., <˜10 W/m²K in a disclosed embodiment). A high thermal conductivity k (e.g., ˜200 W/mK) of the heat sink material (e.g., a 6000 series of aluminum), very low Biot numbers (e.g., <0.1) may be achieved during heat sink analyses, thus negating the requirement of a temperature distribution analysis of the fins.

A natural convection analysis may involve calculation of a film temperature, as in Equation 10:

$${T}_{f} = \frac{{T}_{∞} + {T}_{s}}{2} \qquad Equation 10$$

Properties of air may then be determined at the film temperature, as are shown as approximated in the exemplary figures of Chart 1:

CHART 1
Properties of air at the film temperature, T_{f} = 130° F. = 55° C.

| Property | Value |
|---|---|
| k | 0.03 W/m · K |
| υ | 1.8 · 10⁻⁵ m²/s |
| Pr | 0.7 |

Rayleigh number Raₛ may be determined using air properties at the film temperature and individual heat sink geometries, using Equation 11:

$${Ra}_{s} = {Gr}_{s} Pr = \frac{ℊ β ( {T}_{s} - {T}_{∞} ) {S}^{2}}{{υ}^{2}} Pr \qquad Equation 11$$

Using this Rayleigh number, Nusselt number, Nu, and a correlative heat transfer coefficient can be obtained using Equation 12:

$$Nu = \frac{hS}{k} = {[ \frac{576}{{( {Ra}_{s} S / L )}^{2}} + \frac{2.873}{{( {Ra}_{s} S / L )}^{0.3}} ]}^{- 0.5} \qquad Equation 12$$

Contributions to heat dissipation due to radiation may be determined using the Stefan-Boltzmann law, as set out in Equation 13:

\dot{Q}_{rad}=εσAₛ(Tₛ⁴−T_{∞}⁴) Equation 13

Emissivity ε may be assumed to be 0.9, a typical value for the common heat sink material of black anodized aluminum. Hot side surface area calculations may be made using computer modeling software, such as SolidWorks, to model each heat sink. Finally, the effective heat sink thermal resistance may be calculated using Equation 14:

$$R = \frac{1}{{hA}_{fins}} + \frac{1}{ɛ σ {A}_{s} ( {T}_{s}^{2} + {T}_{∞}^{2} ) ( {T}_{s} + {T}_{∞} )} \qquad Equation 14$$

In some embodiments, analysis of heat sinks of suitable geometry to fit on a prosthesis (e.g., in view of particular size and weight constraints for a given prosthesis) may result in a determination that a passive heat sink would not be adequate to meet the cooling load required for the prosthesis cooling system. For example, it may be determined that a passive heat sink may be insufficient to provide the necessary maximum thermal resistance of approximately 2.7° C./W as calculated above. In some embodiments, therefore, an active heat sink may be used rather than a passive heat sink, such as a heat sink providing 2.9° C./W. A power source may be suitably selected to power both the TECs and active heat sink.

Similar analysis may be made for other types of residual limbs. For example, an above-knee (AK) amputee, such as a person having a transfemoral amputation, may have significantly more muscle mass in the residual limb, thus involving a greater cooling load. For example, in one embodiment, an AK residual limb may be calculated to require approximately 13 watts of cooling. The size and number of TECs may be selected based on the condition of the residual limb and the expected activity level of the patient. For example, for an AK residual limb with substantially all of the muscle groups remaining, the largest muscle masses (commonly referred to as the quadriceps and hamstrings) may lie at the anterior and posterior of the residual limb, respectively. As noted above, optimal cooling may suggest placement of TEC in a prosthesis socket over the centerline of the muscle mass, but TEC cooling efficiency may be balanced against accommodation of patient activity, among other things. For example, as noted above, placing a TEC at the anterior and posterior centerlines of a socket may result in interference with certain basic patient activities, such as sitting, and moving from a standing position to a seated position, and back to a standing position. TEC placement at such positions may cause the TECs to interfere to an undesirable degree with furniture, patient comfort and clothing, particularly if the TEC is used in connection with an active heat sink.

Accordingly, one or more TECs may be placed so as to accommodate expected patient activity. In one embodiment, TECs may be offset from muscle mass centerlines. As may be seen in the AK embodiment of FIG. 12, for example, three offset TECs may be used to carry the cooling load rather than two centered TECs. The three TECs may be offset from main heat generation areas of the residual limb (thigh). In the disclosed embodiment, the first TEC may be placed at or near the line between the rectus femoris and vastus lateralis. The second TEC may be placed at or near the line between the rectus femoris and the vastus medialis. The third TEC may be placed at a posterior location about the biceps femoris and the femoral artery.

The offset may be selected by a prosthetist at the time of socket construction after considering the shape and condition of the residual limb. As may be understood, the human body may vary significantly from person to person, and the shape and condition of residual limbs will equally vary. It should be within the expertise of a skilled prosthetist to construct a suitable socket for a given residual limb, and to determine, in consultation with a patient's physician, the appropriate level and types of patient activity and corresponding cooling load. A prosthesis cooling system may be designed and implemented according to the teachings and principles of this application to account for any required trade-offs between optimal cooling arrangements, residual limb condition and patient lifestyle.

Thus, in the foregoing embodiment, an approximate 13-watt cooling load may be divided generally equally among the three TECs. Each TEC may be required to remove at least 4.3 watts of heat from the residual limb. Accounting for TEC inefficiency, for example, three of the above-mentioned 25.7-watt, 3.0 Aₘₐₓ, 15.4Vₘₐₓ TECs may be selected to accommodate heat removal over a range of activities and environments. An active heat sink may be used for each TEC, such as a heat sink capable of accommodating a base temperature of 150° F. (65.6° C.), heat Transfer of 18 W, and having thermal resistance of 2.26 W/° C. at approximate room temperature (25° C.). A 30 Ah battery system (such as an 8-cell LiPo battery configuration) may be provided to power the TECs, and may provide up to five continuous hours of power. Thus, by dividing the cooling load between a plurality of TECs, a prosthetist may be able to construct a cooling system without having to stock a wide range of TECs, power supplies and heat sinks The voltage boosters and most of the passive elements of the PCB may be designed accordingly. Of course, a PCB may be configured to accommodate a range of TECs and power supplies. For an AK prosthesis cooling system, the increase in weight and size of the battery system can easily be accommodated at a location above the knee. Moving the bulk of the battery system above the knee may provide a lower moment arm than locating at the ankle. With the greater strength of the upper leg as compared to the lower leg, the increase in weight may also be acceptable. Of course, a pylon for an AK prosthesis may include an articulating joint, and placement and configuration of the power source and TEC leads may be made so as to not interfere with such articulation.

In yet other embodiments, one or more TECs may be used to cool residual upper limbs, such as those for above the elbow and below the elbow amputees. TECs may be placed so as to provide limb cooling while reducing interference with patient activities. Cooling load analysis similar to that described herein for lower limbs may be undertaken for upper limbs. A skilled prosthetist may place the one or more TECs at suitable locations in the prosthesis socket for the residual limb.

In some embodiments, a phase-change material (PCM) may be used to assist in removing waste heat from the prosthesis and TEC. A PCM may be, for example, an inorganic medium such a salt hydrate. PCM may be provided in packet form.

The prosthesis socket may include an insulated enclosure for housing a PCM packet. When the PCM packet has reached its maximum heat storage capacity, it may be removed from the insulated enclosure. A new PCM packet may then be inserted into the enclosure to continue the cooling process. A PCM may be, for example, a cold pack stored in a freezer until use, and may provide approximately ½ hour of cooling per packet in some embodiments.

In some embodiments, a PCM packet may be housed in an insulated enclosure that is embedded into the wall of the socket at the time of the socket's fabrication. As may be seen in the embodiment of FIG. 8, a packet enclosure 800 may comprise insulating walls 804 that insulate the PCM packet 802 from the outside environment. The enclosure walls 804 may be of a material that has very low thermal conductivity to create the desired insulating effect.

In some embodiments, the walls 804 of the enclosure may terminate in a flange 806 that may be overlaid with carbon fiber layer of the socket, as may be seen in the example of FIG. 9. Use of a flange 806 may better allow the enclosure to be embedded into the socket and thus become a permanent structural component of the socket. The enclosure wall 804 may be hinged 808 and have a locking mechanism 810 (e.g., hook-and-loop fasteners, snaps, friction fitment, interlocking tabs, pins, or other suitable means) to allow PCM packets 802 to be retained and removed as desired. The enclosure 800 may be open to the TEC 812 hot side surface 814 or a heat sink (not shown), so as to allow thermal communication between the TEC element and the PCM packet.

Any suitable TEC may be used, including those having metallic, ceramic, flexible thin film or other surface materials. A TEC may rely on the Peltier effect in which an electrical current is applied to induce a heat flux through two dissimilar conductive materials. In some embodiments, a TEC may comprise two ceramic plates separated by a thermally-conductive substrate, which may comprise thermoelectric couples (N- and P-type semiconductor legs) connected electrically in series and thermally in parallel. One plate absorbs heat and the other plate rejects heat. The heat flux through the TEC may be directed to a heat sink and rejected to atmosphere.

The TEC may be powered by any suitable power source. In some embodiments a battery pack may be used, such as a 7 Ah or 11 Ah rechargeable lithium polymer battery pack. As may be seen in the embodiment of FIG. 1, a power source may be mounted to the prosthesis pylon or any other suitable location, and may be connected to the TEC by wire leads. The leads may be run through the prosthesis socket, or routed externally to a power source. The power source may be suitably sized to run the TECs and any active cooling devices for a suitable length of time. If only passive cooling is used (e.g., radiation and natural convection from a heat sink), power requirements may be reduced.

In some embodiments, a fuel cell, such as a proton exchange membrane fuel cell, may be used as a power source. In some embodiments, a hydrogen fuel cell may be provided as a primary or secondary power source. A suitable fuel cell may be selected for ease of installation. For example, many off-the-shelf fuel cells are designed with a USB connector, such as an Intelligent Energy's Upp brand fuel cell, that may permit easy replacement in a prosthesis cooling system. The connection point between the control circuit and fuel cell in such embodiments may thus be a USB male connector that has leads connected to a control circuit PCB, such as described below.

In other embodiments, one or more piezoelectric generators may be disposed at various prosthesis flex points, such as in the pylon, or at flex regions of a prosthetic foot. A piezo-electric generator may implement the piezoelectric effect to generate electricity where a difference in stress exists. Such a generator may employ piezoelectric plates oriented to absorb a deflection through human movement to generate electricity. In some embodiments, the generator may incorporate one operational surface area where a difference in stress between the body and the plate exists. In the configuration, the device may be applied in close proximity to the human body and permitted to interface with any zone where a deflection may be obtained. Upon generating a stress gradient, the deflection in material used for a piezoelectric generator may generate an electromotive force that may be delivered to a storage media such as a battery or capacitor for later use or for immediate recharge of a power supply.

A piezoelectric generator may comprise a piezoelectric element, such as polyvinylidene fluoride (PVDF), sandwiched between thin metal electrodes. Wire leads may be routed from the electrodes to control circuitry that manages battery recharging. A control circuit may include a battery charge controller integrated circuit and battery fuel gauge integrated circuit. The control circuitry may control battery charge and discharge.

Power may be controlled by any suitable circuitry. For example, a battery control circuit may be used to adjust the power source voltage to a voltage suitable for the TECs and active cooling devices. For example, power voltage adjustment may be accomplished using DC-DC converters. The circuit may comprise a switch to turn the system on and off. A battery control circuit may regulate battery discharge, and may monitor battery temperature. A battery control circuit may adjust battery usage to maintain battery temperature within a safe temperature range. A battery control circuit may include a visual indicator of battery charge level.

In some embodiments, a PCB may be used that includes charging control elements for a battery, voltage boosters, a manual SPST switch, passive elements for conditioning the power signal, connectors for attaching TEC leads, and a power source recharge or connection port. A suitable enclosure may be provided for the PCB, such as a weather- and impact-resistant casing. The enclosure may also house a battery or other power source. In some embodiments, various connectors and switches may be weather resistant, and the PCB may be conformal coated.

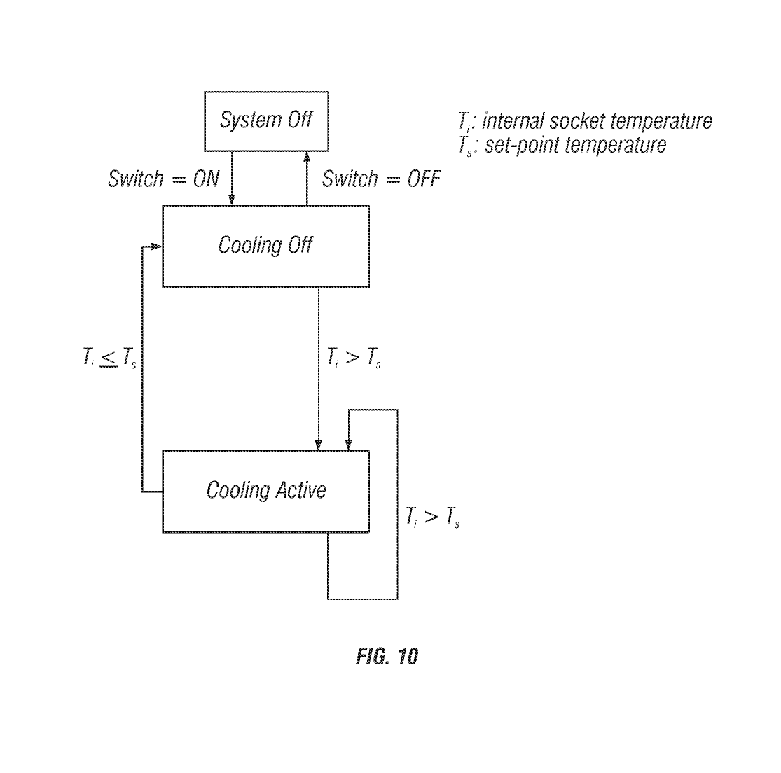

A closed-loop temperature control system may be provided. A manual cut-off SPST rocker switch may be provided for user override. A suitable closed-loop control system may include a microcontroller linked to one or more temperature sensors that have been embedded at various locations in the socket, such as in the acrylic substrate of a carbon fiber socket. Temperature sensors may include thermistors, thermocouples, resistance temperature detectors (RTDs), and the like. As may be seen in a state transition embodiment of the cooling system of FIG. 10, the microcontroller may activate the cooling system when the interior socket temperature (Tᵢ) reaches or exceeds a set point temperature (Tₛ). The system may continue to cool as long as Tᵢ>Tₛ and the user has the switch in the ON position. When Tᵢ<Tₛ, the system may deactivate until otherwise needed.

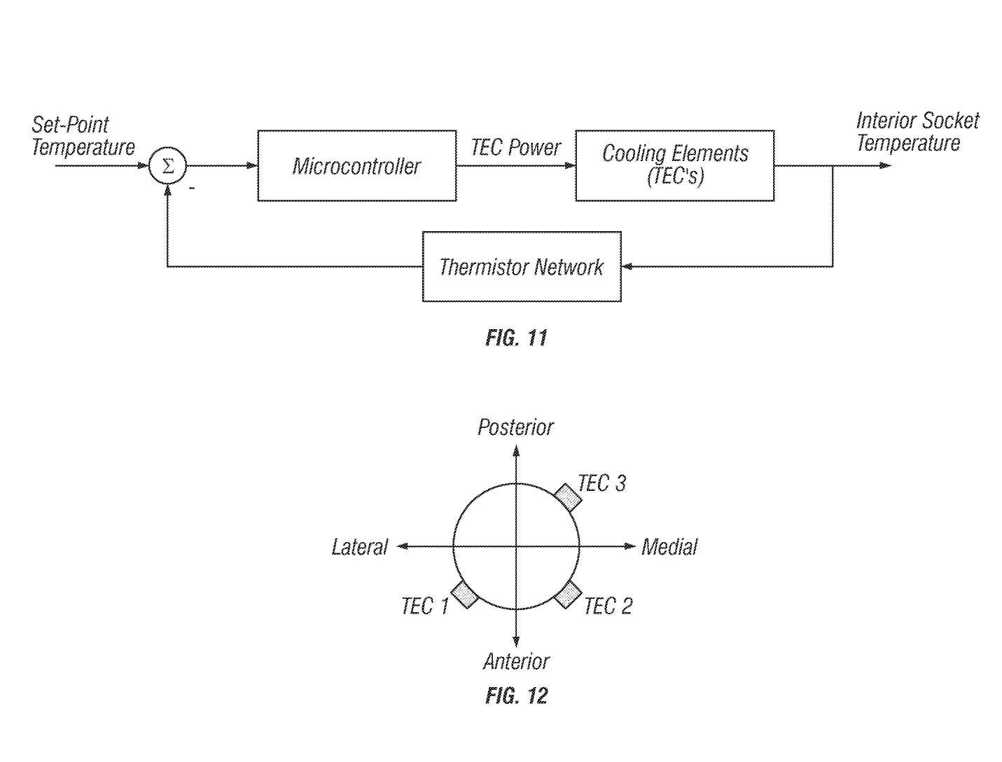

The microcontroller may function on a proportional-integral (PI) control system. Such a control scheme may eliminate the error function as in conventional PID control theory, yet sacrifice response time. As temperature change may function on a slow response curve with the physical configuration of the system, the response time of the controller may be sacrificed in favor of simplicity. An example functional block diagram of a control system may be seen in FIG. 11.

Furthermore, by dividing the cooling load among a plurality of TECs, the cooling load may be balanced or allocated among the TECs to optimize cooling, patient comfort and battery life. In some embodiments, a closed-loop temperature control system may selectively activate a TEC based on local socket temperatures. Thus, a TEC located over an area of lower heat generation may be cycled off to allow power to be used by a TEC over an area of higher heat generation. As may be understood from the teachings of this application, one or more TECs may be placed at any suitable location of the prosthesis socket.

Although the disclosed subject matter and its advantages have been described in detail, it should be understood that various changes, substitutions and alterations can be made herein without departing from the invention as defined by the appended claims. Moreover, the scope of the claimed subject matter is not intended to be limited to the particular embodiments of the process, machine, manufacture, composition, or matter, means, methods and steps described in the specification. As one will readily appreciate from the disclosure, processes, machines, manufacture, compositions of matter, means, methods, or steps, presently existing or later to be developed that perform substantially the same function or achieve substantially the same result as the corresponding embodiments described herein may be utilized. Accordingly, the appended claims are intended to include within their scope such processes, machines, manufacture, compositions of matter, means, methods or steps.

1. A prosthesis cooling system for a BK amputee, the system comprising:
a standard prosthesis socket configured for attachment to a BK residual limb;
a first thermoelectric cooling element (TEC) configured for embedding in the prosthesis socket at a location generally corresponding to a first apex of the gastrocnemius muscle;
a second TEC configured for embedding in the prosthesis socket at a location generally corresponding to a first apex of the gastrocnemius muscle; and
a power source electrically coupled to each TEC.

2. The system of claim 1 further comprising a power control circuit.

3. The system of claim 1 further comprising a closed-loop temperature control system.

4. The system of claim 1, wherein the power source is one of a battery and fuel cell.

5. The system of claim 1, wherein the power source is a battery, the system further comprising a piezoelectric generator coupled to the power source, the power source being configured for mounting at a flex point of the prosthesis.

6. (canceled)

7. The system of claim 1,
the socket comprising an external layer forming a first aperture configured to receive the first TEC and forming a second aperture configured to receive the second TEC, an internal layer, a first channel extending from the first aperture to the exterior of the socket, and a second channel extending from the second aperture to the exterior of the socket; and
the first TEC and second TEC each comprising a cold side surface, a hot side surface and leads, wherein the first TEC is disposed in the first aperture and the second TEC is disposed in the second aperture, such that the cold side surface of each TEC is in a conductive heat exchange relationship with the internal layer and the hot side surface is disposed away from the internal layer, and the leads of the first TEC are disposed in the first channel and the leads of the second TEC are disposed in the second channel.

8. (canceled)

9. The system of claim 1, wherein the first apex is the medial apex and the second apex is the lateral apex.

10. A limb prosthesis comprising:
a standard socket comprising a first layer and a second layer, the second layer comprising an exterior surface of the socket, the second layer forming an aperture;
a thermoelectric cooling element (TEC) comprising a cold side surface, a hot side surface and leads, wherein the TEC is disposed in the aperture such that the cold side surface is in a conductive heat exchange relationship with the internal layer and the hot side surface is oriented away from the internal layer;
a heat sink mounted to the hot side surface; and
a power source electrically coupled to the TEC.

11. The system of claim 10, the first layer comprising acrylic and the second layer comprising carbon fiber.

12. (canceled)

13. (canceled)

14. (canceled)

15. (canceled)

16. (canceled)

17. A prosthesis cooling system for an AK amputee, the system comprising:
a standard prosthesis socket;
a first thermoelectric cooling element (TEC) disposed in the socket wall at a location generally corresponding to a location between the rectus femoris and vastus lateralis;
a second TEC disposed in the socket wall at a location generally corresponding to a location between the rectus femoris and the vastus medialis;
a third TEC disposed in the socket wall at a location generally corresponding to a location near the biceps femoris and the femoral artery; and
a power source electrically coupled to each TEC.
